(19) United States
(12) Patent Application Publication
(10) Pub. No.: US 2026/0315165 A1
(43) Pub. Date: Oct. 15, 2026

(54) ASSISTANCE SYSTEM, COMPUTER-IMPLEMENTED CONTROL METHOD, AND COMPUTER-READABLE STORAGE MEDIUM

(71) Applicant: B. Braun New Ventures GmbH, Freiburg im Breisgau (DE)

(72) Inventors: Tim Beyl, Freiburg im Breisgau (DE); Amir Sarvestani, Freiburg (DE); Jean Stawiaski, Freiburg (DE)

(21) Appl. No.: 19/483,800

(22) PCT Filed: May 16, 2024
(86) PCT No.: PCT/EP2024/063603

§ 371 Date: Nov. 12, 2025

(30) Foreign Application Priority Data
May 17, 2023 (DE) 10 2023 113 045.5

(51) Int. Cl. A61B 34/32; A61B 34/20; A61B 90/00

(52) U.S. Cl. CPC A61B 34/32; A61B 2034/2055; A61B 2034/2065; A61B 2090/3937

(57) ABSTRACT

A surgical assistance system includes a surgical robot having a movable robot arm, a visualization system designed to create at least one real-time image, a surgical effector having a working point arranged on a working axis of the surgical effector, and an optical reference marker arranged on the surgical effector. The working point can be positioned in a field of view of the visualization system. The surgical effector is designed to allow a position of the working point and/or an orientation of the working axis to be determined. A computer-readable storage medium contains instructions to perform a method for controlling the visualization system.

### CROSS-REFERENCE TO RELATED APPLICATIONS

This application is the United States national phase entry of International Application No. PCT/EP2024/063603, filed on May 16, 2024, and claims priority to German Application No. 10 2023 113 045.5, filed on May 17, 2023.

### FIELD

The present disclosure relates to a medical, in particular surgical, assistance system, in particular a neurosurgical assistance system, comprising a navigated surgical robot for use in a surgical intervention on a patient. For this purpose, the robot has a robot base as a local link point of the robot and in particular as a local, stationary coordinate system or reference coordinate system. A movable or actively movable robot arm with at least one robot-arm segment is connected to the robot base. In addition, the assistance system has a visualization system as its end effector, in particular with one or more cameras, which is attached, in particular mounted, to the robot arm, in particular to an end side of the robot arm. The visualization system is adapted to create at least one real-time, corporeal, preferably intracorporeal, image of the patient and to provide it, preferably digitally. In addition, the present disclosure relates to a computer-implemented control method and a computer-readable storage medium and a computer program.

### BACKGROUND

In the field of medicine and medical technology, automation with the associated integration of digitally controllable technical devices is becoming increasingly important.

Robots are increasingly being used for surgical interventions, which in particular support precise minimally invasive interventions. Here, the robot is not only provided as a stand-alone robot that only performs the operation, but the robot is increasingly used as a collaborative robot (cobot), i.e. as an assisting or supporting robot, directly in the intervention field, which interacts with a medical professional, in particular the surgeon.

For such robot-assisted surgical interventions, in particular neurosurgical interventions on the brain, various visualization and measurement systems are generally used for visual, physiological and functional assessment of the intervention region and as a decision-making aid. The classic data detected during the intervention include the images from a visualization system placed in the intervention region, for example a surgical microscope and/or endoscope.

In conventional assistance systems, the instrument is tracked via optical reference markers attached to the instrument, in particular by a rigid body with four spheres that can be tracked in space. In neurosurgery, either a navigation pointer or a suction tube is used as an instrument. However, reference markers influence and impair ergonomics due to their size and weight, cause additional effort and costs and are complex to use. They also cause line of sight problems, since the microscope often blocks the view between the navigation camera and the instrument, and the rigid body with the reference markers also obscures the view in the microscope's field of view. This then requires a manual correction movement of the navigation camera, which invalidates an automatic follow-up function, i.e. automatic trailing movement of the visualization system depending on the instrument movement. An additional problem is that the optical axis of the visualization system is coincident (coaxial) with the working axis of the instrument. As a result, the field of view of the visualization system is strongly influenced by the shaft of the instrument and is partially obstructed by it.

### SUMMARY

In view of this, the object of the present disclosure is to avoid or at least reduce disadvantages of the prior art and in particular to provide a surgical assistance system, a computer-implemented control method, a computer-readable storage medium, and a computer program which provides a particularly good and intuitive control of a visualization system. A partial object can be seen in providing a hands-free control system in which the surgeon can continue to use both hands for the intervention, but can also perform the control with instruments in his/her hand. A partial object can be seen in using the simplest possible objects to control the robot for visualization during an intervention, wherein these objects in particular do not obstruct the view of a visualization system. The aim is also to create a way for a user to control the robot by adapting it as little as possible, thereby improving manufacturing costs and avoiding sources of error in existing effectors, such as medical instruments. Another object can be seen in the fact that an effector may be used to control the robot and the visualization system with particular precision. Ergonomics should also be improved.

Thus, a basic idea of the disclosure is to provide a surgical assistance system comprising a robotically moved visualization system comprising in particular a surgical microscope or an endoscope—or both—and which is adapted to control the movement of the visualization system via a (medical or surgical) effector, in particular a surgical instrument, wherein (image-based) picture-based tracking is used to control, in particular track, an optical axis of the visualization system relative to a working axis of the effector, wherein this control or tracking is continuously/permanently active and/or may be activated by a user selectively, in particular on demand.

Specifically, a surgical assistance system, in particular a neurosurgical assistance system, comprises a surgical robot, in particular a navigated surgical robot, for use in a surgical intervention. The robot has a robot base as a local link point of the robot and a movable robot arm connected to the robot base with at least one robot-arm segment, in particular a plurality of robot-arm segments, which are connected to each other via bearings, in particular hinges, and which enable an active configuration of the robot arm. A visualization system (or a visualization unit) is attached, is in particular mounted, to the robot arm, in particular to one end of the robot arm, as an end effector. This visualization system is adapted to create at least one real-time, intracorporeal image of the patient and to subsequently provide it digitally and thus in a computer-readable manner. The assistance system furthermore includes a surgical effector (separate from the robot), in particular a (separate) surgical instrument, wherein the effector may be manually guided in particular by a medical professional or is guided or mechanically activated or held. A working point of the effector is arranged on or at a main, longitudinal, or working axis of the effector. As intended, the working point is arranged or at least arrangeable in a field of view of the visualization system, so that the visualization system provides the surgeon with the intracorporeal image with the working point depicted therein as required.

Furthermore, an optical reference marker is provided, which is arranged on the effector in a predetermined position with respect to the working point-in particular with a predetermined distance, in particular with a predetermined position and orientation. The reference marker is adapted so that a position of the working point and/or an orientation of the working axis may be determined on the basis of its optical detection. According to the disclosure and in contrast to conventional optical reference markers in the sense of a “rigid body”, the reference marker is formed by an optical pattern which extends along at least a surface portion of the effector, preferably along a surface portion of an effector housing. In principle, the pattern may be located at any position on the effector, but a distal area or portion is preferred, in particular in the vicinity of the working point, in order to enable particularly precise localization in the field of view of the visualization system. This distal placement of the pattern allows, in particular, that instruments such as suction devices, suction cups or suction tubes can be bent before use in order to adapt them to the anatomy and ergonomics without affecting the navigation accuracy. Another component of the assistance system is a tracking system, in particular a navigation system, which is adapted according to the disclosure to determine the position of the working point and/or the orientation of the working axis as a function of or based on the detected pattern. In particular, the adaptation of the tracking system consists in the fact that it has an algorithm for machine vision via which the position of the working point and/or the orientation of the working axis may be determined on the basis of the image and the pattern contained therein in relation to a coordinate system of the visualization system. A control unit of the assistance system is adapted to control a position of the visualization system and/or an orientation of an optical axis of the visualization system and/or a hyperfocal distance of the visualization system as a function of the determined position of the working point and/or of the determined orientation of the working axis. The tracking system of the assistance system may preferably also be used, in particular with spatial reference to the patient, to track the pose, i.e. a position and an orientation, of the visualization system, and thus the pose of the at least one real-time, corporal image.

The extension of the reference marker configured as a pattern along the surface or the surface portion of the effector, which is in any case arranged or to be arranged as intended in the field of view, has the advantage over a conventional, optically detectable reference marker, such as a separate fixed body/rigid body, which projects beyond the effector or its effector housing, that the reference marker may no longer be restricted in its visibility by the visualization system—for example the microscope—so that it is no longer necessary to reposition the microscope, and that the field of view of the visualization system may no longer be blocked by the rigid body. The visualization system therefore no longer has to be adapted in its position and orientation to an interfering position or pose of a reference marker, as is necessary in the prior art when, for example, the rigid body comes into the field of view.

The term “effector” in the present disclosure means, for example, an apparatus, an instrument, or similar medical means which may be used in an intervention for performing the intervention. In particular, an effector may be considered to be: an instrument, a medical apparatus such as an endoscope or a suction tube, an optical apparatus with a visualization axis, a pointer/indicator with a distal tip for surgical navigation and others. The “working axis” of the effector means here in particular a longitudinal axis of the effector, in particular of a distal portion of the effector, such as a longitudinal axis of an area of the cutting portion of a scalpel or a longitudinal axis of an endoscope tip or, in the case of a rigid endoscope, also the longitudinal axis of the endoscope shaft. The working axis of, for example, a dental tool with a non-rectilinear distal working portion and working point, such as a probe, may also be defined in the area of the rectilinear portion of the instrument of the handle.

The term “working point” here refers to the point or area of the effector that is used as intended during an intervention. For example, the working point of a scalpel is the tip of the scalpel, the tip of the pointer of a pointer/indicator, the tip of the sheath of scissors in the closed state, the tip of the tweezers of tweezers, the tip of a drill or cutter, the tip of a syringe, the tip of a trocar, the tip of the endoscope for an endoscope, while in the case of a dental tool of a probe, the working point is the tip of the instrument, which (slightly offset) from the working axis is not the most distal point but runs slightly “back” in the proximal direction, similar to a hook.

The term “robot-arm segment” here means in particular a robot part of the robot arm mounted between bearings or hinges or, in the case of the end-sided robot-arm segment, in particular a robot part connected in series between the end effector and preceding robot-arm segment (correspondingly the robot base in the case of only one robot-arm segment).

The term “position” means a geometric position in three-dimensional space, which is specified in particular via coordinates of a Cartesian coordinate system. In particular, the position may be specified by the three coordinates X, Y and Z.

The term “orientation”, on the other hand, indicates an alignment (e.g. at the position) in space, in particular that of an axis. It can also be said that the orientation specifies an alignment with indication of direction or rotation in three-dimensional space. In particular, the orientation may be specified using a maximum of three angles.

The term “pose” includes both a position and an orientation. In particular, the pose may be specified via six coordinates, three position coordinates X, Y and Z and a maximum of three angular coordinates for the orientation.

Advantageous embodiments are explained in particular below.

According to a further development, the control unit is adapted to move the visualization system in a tracking mode such that the position of the working point of the effector coincides with a center point of the image or with another predetermined or predefined point of the image, so that the visualization system follows the working point of the effector, and that the optical axis has a predetermined angle, in particular a static relation, to the working axis and maintains this angle during movement of the effector. In this tracking mode, which in particular may be activated and deactivated by the user, the effector with its working axis may be used like a joystick to set an angle and perform spatial positioning. For example, a tip of a pointer as a working point may indicate which point should be the center of the image and the robot moves the visualization system accordingly and follows the tip of the pointer. In addition, an alignment of the optical axis is also specified via the longitudinal axis of the pointer as the working axis with a predetermined angle, for example 45°. This allows the user to intuitively and quickly target an area, in particular using a surgical microscope as a visualization system, and to display it without interrupting the operation and without having to use their hands for adjustment. Due to the optical reference marker, the instrument, such as the pointer, only needs to be changed slightly, in particular no new approval is required, since only a surface is optically marked, and the adaptive control unit may use the instrument as an input means.

According to a further development, the control unit is adapted to determine a flat band, in particular a spanned plane, spanned by an, in particular initial, optical axis of the visualization system and by an, in particular new, working axis of the effector, and to define a new optical axis of the visualization system with a predetermined angle to the new working axis in this flat band/plane (upon movement of the effector and corresponding change of the working axis), and to control the robot arm for an arrangement of the visualization system in space accordingly.

The surface portion of the effector with the pattern is preferably curved, alternatively it is planar. The pattern is preferably a 3D object, alternatively it is a 2D object. The pattern is preferably formed by an imprint or an anodization of the surface portion or by a laser application. Mixed forms of these are of course possible.

Preferably, the surface portion has a shape based on a standard geometry, preferably a cylindrical shape. Preferably, its cross-section is circular or polygonal, in particular quadrangular or pentagonal or hexagonal. In a preferred further development, the pattern is formed parallel to the surface portion. Alternatively, the pattern has a cross-section with respect to the working axis that differs from the cross-section of the surface portion, for example a polygon-shaped cross-section, while the surface portion has a circular cross-section. In this case, the pattern forms angled portions that may be easily evaluated in the image using image recognition. The pattern is at least partially point-shaped, and/or linear, and/or curved.

According to a further development, the effector is configured with a fixed, i.e. non-interchangeable functional unit. According to the disclosure, a functional unit is a tool that fulfills the required function during the intervention. It is, for example, a cutter, a drill or an imaging system, such as an ultrasound probe, or the like. The working point is located distally (at the tip), or at least in a distal area, of the functional unit.

Alternatively, the effector has an interchangeable holder into which one or one of several possible functional units or tools may be selectively inserted. The pattern is preferably arranged in an area of the interchangeable holder. Preferably, all functional units, when inserted into the interchangeable holder, have the same distance and preferably the same orientation of their working point to the pattern arranged on the interchangeable holder. When changing the functional unit, there is then no need to enter information into the assistance system about any deviating distance or orientation of the working point.

In order to ensure the detection of the pattern and the determination of the position of the working point and/or of the orientation of the working axis based on it at all times, according to a preferred further development, the pattern is configured and/or dimensioned and/or arranged on the surface portion in such a way that it is arranged at least in portions in the field of view when the working point is arranged in the field of view as intended.

According to a further development, the pattern is configured in such a way that it is still detectable as intended—i.e. for determining the position of the working point and/or of the orientation of the working axis—even if it is partially obscured, for example by the operator's hand.

According to a further development, the pattern has a variation at least in the direction of the working axis.

According to a further development, a model of the pattern is stored in the tracking system or is provided to the tracking system by the control unit. Preferably, the tracking system is adapted to determine the position of the working point and/or of the orientation of the working axis of the effector as a function of the detected pattern and of the stored model.

In particular, the image, in particular the microscope image, is used to detect the position and/or orientation of the instrument in the microscope coordinate system via machine vision, i.e. vision via a machine. Preferably, the microscope and the patient are detected by an external camera, in particular that of a navigation system, whereby a spatial tracking chain is closed and the position and/or orientation of the instrument in a patient coordinate system may be calculated via the control unit.

If the detection of a rotation of the effector around its working axis is desired or relevant for the control of the visualization system, the pattern also has a variation in the circumferential direction to the working axis according to a further development.

The variation may be of a geometric and/or color nature in particular.

In order to achieve the best possible detectability of the pattern independently of a rotation of the effector, the pattern according to a further development extends completely around or at least circumferentially around the working axis.

However, in the case that the rotation of the effector is not relevant, i.e. if, for example, the visualization system is not to follow a rotation of the effector, a further development in which the pattern is at least partially rotationally symmetrical to the working axis proves to be advantageous. If, on the other hand, the rotation of the effector is relevant, in particular if the visualization system is to follow the rotation of the effector, the pattern is at least partially rotationally asymmetrical to the working axis.

In a preferred further development, the pattern has elements that are spaced apart in the direction of the working axis.

In particular, the effector may have concentric rings as a pattern around its working axis that are spaced apart from each other and have a different width in the direction of the working axis.

In order to be able to easily determine the position of the working point and the orientation of the working axis even when the pattern is partially obscured, according to a further development, a distance of the elements from each other and/or a respective extension of the elements in the direction of the working axis has a variation.

With the same effect, according to a further development, at least some of the elements are combined to form groups within which the elements are the same with respect to their distance from each other and/or their respective extension in the direction of the working axis, while the distance of the elements from each other and/or the extension of the elements across the groups has or have a variation.

According to a particularly preferred further development, the visualization system is adapted to provide the image containing the pattern at least in portions to the tracking system, wherein the tracking system is adapted to determine the position of the working point and/or the orientation of the working axis of the effector as a function of the image. The advantage is that the visualization system thus has a dual function. On the one hand, it provides the image required by the surgeon for visualizing the surgical site. On the other hand, it uses the image to provide the necessary input for controlling its position, and/or orientation, and/or focal distance that depends on the effector.

In order to determine this, the tracking system preferably has an image recognition algorithm that is adapted to determine the position of the working point and/or of the orientation of the effector as a function of the image of the visualization system. Alternatively or additionally, the tracking system has a machine learning algorithm for image recognition, which is adapted to determine the position of the working point and/or of the orientation of the effector depending on the image of the visualization system.

This means that separate tracking or navigation cameras no longer need to be provided in order to determine the position of the working point and/or pose of the effector. This is simpler in terms of device technology, incurs lower costs, and requires less installation space, thus increasing the space available in the operating room. Of course, at least one separate 2D or 3D camera may also be provided for optical detection of the pattern in order to further increase the process reliability or accuracy for determining the position of the working point and/or of the orientation of the working axis. The at least one separate 2D or 3D camera may also be used to navigate the robot arm in relation to the patient.

For the same reason of improving process reliability or accuracy, supplementary detection technologies based on alternative reference markers, for example electromagnetic, X-ray, radar, laser and/or lidar-based technologies, may be provided according to an embodiment.

According to a further development, the control unit is adapted to constantly or continuously control the visualization device with respect to its position and/or orientation of its optical axis and/or its hyperfocal distance. Alternatively, the control unit may be adapted to perform a corresponding control only on request or only after an input. A third variant is that control is performed selectively, i.e. either continuously or on demand, i.e. in two freely selectable modes. This provides both continuous and input-based tracking, between which a user may switch.

According to a further development, a manually operable or an automated request device may be provided, which is adapted to trigger the request. In particular, the following may be mentioned as a requesting device: a foot pedal and/or a push button, which is provided on the effector, for example, and/or a voice recognition device and/or a gesture recognition device.

The position and/or orientation is preferably controlled by driving the robot and its at least one robot arm in such a way that it performs the necessary translation(s) and/or rotation(s) in order to move the visualization system to the position and/or orientation dependent on the effector.

Preferably, the control unit is adapted to control the position of the visualization system and/or the orientation of the optical axis of the visualization system and/or the hyperfocal distance of the visualization system as a function of the determined position of the working point and/or of the determined orientation of the working axis such that the working point is located in a center or a center point or in a predefined point of the field of view, and/or the optical axis is set to a predetermined angle to the working axis, and/or the working point coincides with the hyperfocal distance.

According to a further development, the control unit is adapted to set the hyperfocal distance to the working point when the position of the visualization system is fixed, i.e. unchangeable. Additionally or alternatively, the control unit is adapted to set the position of the visualization system such that the hyperfocal distance coincides with the working point when the hyperfocal distance is fixed. In a preferred further development, the control unit is adapted in both of the aforementioned ways.

A method according to the disclosure is provided for controlling a position and/or an orientation of an optical axis and/or of a hyperfocal distance of a visualization system, in particular of a surgical assistance system, which is configured according to one of the aspects described above. The control is carried out as a function of a tracked surgical effector.

According to the disclosure, the method has the following steps:
“creating and real-time providing of a real-time image of an intervention region, wherein a working point of the surgical effector is arranged or may be arranged as intended in a field of view of the visualization system”. This step is performed via the visualization system, which is connected to a robot arm;
“optically detecting an optical reference marker, or at least a portion thereof, wherein the optical reference marker is formed by an optical pattern which extends along at least a surface portion of the effector and which is arranged on the effector in a predetermined position—in particular a predetermined distance—and a predetermined orientation with respect to the working point, and which is adapted to determine a position of the working point and/or an orientation of the working axis from its optical detection”.
“determining the position of the working point and/or of the orientation of the working axis as a function of the optically detected pattern, in particular via machine vision on the basis of the image in relation to a coordinate system of the visualization system”. This step is performed via a tracking system; and
“controlling the visualization system with respect to its position and/or its orientation of its optical axis and/or its hyperfocal distance as a function of the determined position of the working point and/or of the determined orientation of the working axis”. This step is carried out via a control unit adapted for this purpose.

Preferably, the method also has a step of “tracking, in particular navigation, of the visualization system, preferably of a surgical microscope and/or a surgical endoscope”. This step is performed by the tracking system or alternatively by a navigation system.

According to a further development of the method, the step “controlling the visualization system with respect to its position and/or its orientation of its optical axis and/or its hyperfocal distance as a function of the determined position of the working point and/or of the determined orientation of the working axis” is carried out continuously. Continuous means that the visualization system continuously follows every movement of the effector. A further development is possible in which the movement of the visualization system is damped or smoothed so that the visualization system moves smoothly even if the effector moves jerkily. The same damping or smoothing is of course possible for the adjustment of the focal distance.

Alternatively, control only takes place on request, preferably as a function of a request signal, so that the visualization system is static in terms of position and/or orientation and/or focal distance over a period of time until the request signal occurs and the visualization system is repositioned and/or aligned with the effector and/or its focal distance is adjusted.

The request signal may be triggered manually, for example by the surgeon. Alternatively, the request signal may be triggered automatically, in particular depending on at least one piece of surgical context information.

Furthermore, a further development is possible in which the movement of the robot, and consequently the movement of the visualization system, is restricted to individual translational or rotational degrees of freedom X, Y, Z, RX, RY, RZ, or to a selection thereof, for example only to rotations or only to translations.

According to a further development, the step of “optically detecting the pattern or the portion of the pattern” is carried out by the visualization system by creating the image in such a way that the pattern or at least the portion of the pattern is depicted therein.

According to a further development, the step of “determining the position of the working point and/or of the orientation of the working axis” is performed via an image processing algorithm stored in the tracking system, in particular navigation system, for execution, which processes the image.

According to a further development, the step “controlling the visualization system with respect to its position and/or its orientation of its optical axis and/or its hyperfocal distance as a function of the determined position of the working point and/or of the determined orientation of the working axis” is performed as a function of a deviation of the current position and/or current orientation of the optical axis and/or current hyperfocal distance of the visualization system from a target.

The target includes in particular that the working point is visible in a predetermined area of the field of view, preferably a center or center point or a predefined point, and/or that it is depicted in the image, and/or that the working point coincides with the hyperfocal distance, and/or that the optical axis has a predetermined angle to the working axis. If the predetermined angle is not equal to zero, the effector, in particular its shaft, is prevented from blocking or partially blocking the field of view of the microscope. Preferably, the control unit is adapted in such a way that the predetermined angle can be preset, adapted, and/or changed, preferably depending on the preferences of the operator. Alternatively, the predetermined angle can be 0°.

According to a further development, the step of “controlling the visualization system with respect to its position and/or its orientation of its optical axis and/or its hyperfocal distance as a function of the determined position of the working point and/or on the determined orientation of the working axis” includes a step of “driving the robot at least in such a way that a predetermined area of the field of view or the image, in particular a center, a center point or a predefined point, is moved into alignment with the working point, and that the optical axis is set to a predetermined angle to the working axis”. With regard to the requirement that the working point coincides with the focal distance, there are two possible steps. In one variant, the distance of the visualization system to the working point is fixed and it is followed by “setting the hyperfocal distance so that the working point is focused or coincides with the hyperfocal distance”. In another variant, the hyperfocal distance is fixed and “setting of a distance of the visualization system to the working point in such a way that the working point is focused or coincides with the hyperfocal distance” takes place.

However, the predetermined angle as the sole target cannot define a clear pose of the optical axis in space, since the optical axis may still rotate around the working axis or an axis parallel to it. In order to clearly define the optical axis in space, the plane in which the optical axis actually lies has to be defined. According to the disclosure, there are two ways of doing this:

In one variant, a plane is generated that is spanned by the determined working axis and a focus point of the current optical axis. The target of the new optical axis then lies in this plane with the predetermined angle to the determined working axis. In this variant, the optical axis does not rotate if the effector is rotated around its working axis.

In another variant, the pattern is configured in such a way that all translations and rotations of the effector may be determined from its detection, including the rotation around its own working axis. In this case, the optical axis is moved when the effector is rotated around its own axis.

For a majority of possible effectors or instruments, it may be assumed that the effector's own rotation is not relevant for tracking the visualization system. The first variant is therefore the preferred one.

With respect to a computer-readable storage medium or a computer program, the objects are solved in that the storage medium or computer program comprises instructions which, when executed by the computer, cause the computer to perform the steps of the method according to the present disclosure.

Any disclosure relating to the surgical assistance system according to the present disclosure applies to the method according to the present disclosure as well as vice versa.

### BRIEF DESCRIPTION OF THE DRAWINGS

The invention is explained in more detail below with reference to preferred embodiments with the aid of Figures. The following is shown:

FIG. 1 shows a schematic side view of a surgical assistance system according to a preferred embodiment;

FIG. 2 shows a perspective view of a visualization system and an effector of the assistance system according to FIG. 1;

FIG. 3 shows a detailed view of the effector with an optical reference marker in the form of a pattern according to FIG. 2;

FIGS. 4a-4d show a schematic representation of a sequence of a procedure with a surgical assistance system according to FIGS. 1 to 3; and

FIG. 5 shows a detailed view of FIGS. 4a-4d.

The Figures are schematic in nature and are intended to aid understanding of the invention. Identical elements are provided with the same reference signs. Features of different embodiments may be interchanged.

### DETAILED DESCRIPTION

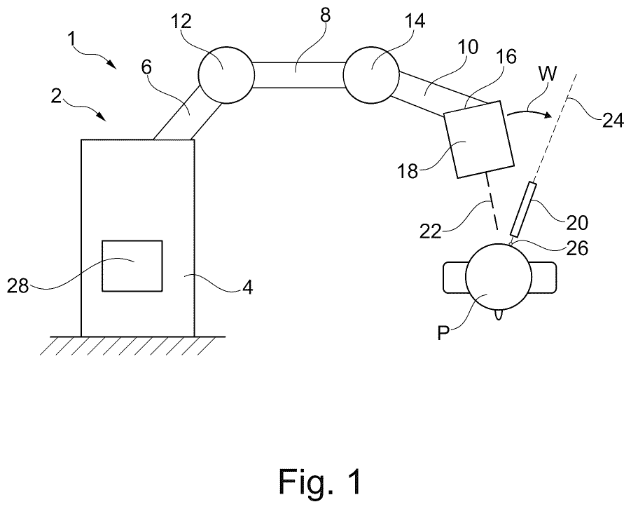

FIG. 1 shows a surgical assistance system 1 according to a preferred embodiment in schematic side view. The assistance system 1 has a surgical robot 2 with a robot base 4, which is locally fixed in the embodiment shown. Alternatively, it may be mobile so that the robot 2 may be used at different locations in a hospital operating theater, for example, as required. In either case, the robot base 4 forms a local reference point to which a multi-segmented robot arm with several robot-arm segments 6, 8, 10 is connected, which are connected to each other via hinges 12, 14. In this way, the robot-arm segments 8 and 10 may be actively moved in relation to each other and the robot arm 6, 8, 10, 12, 14 may be controlled as a whole.

A visualization system 18 of the assistance system 1, which in the embodiment shown is configured as a surgical operating microscope, is attached to one end 16 of the robot arm. In the embodiment shown, the assistance system 1 also has a manually guided effector 20, or an instrument 20, tracked by a tracking system of the assistance system 1. An optical axis 22 of the visualization system 18 is angled at an angle W to a working axis 24 of the effector 20. A working point 26 of the effector 20 is located at the distal end of the working axis 24.

A position of the visualization system 18 and an orientation of its optical axis 22 may be controlled and adjusted via a specially adapted central control unit 28 placed in the base 4, wherein the special adaptation consists in particular in the fact that it is carried out as a function of a position of the working point 26 and/or orientation of the working axis 24 determined in each case via machine vision, which is explained in more detail below.

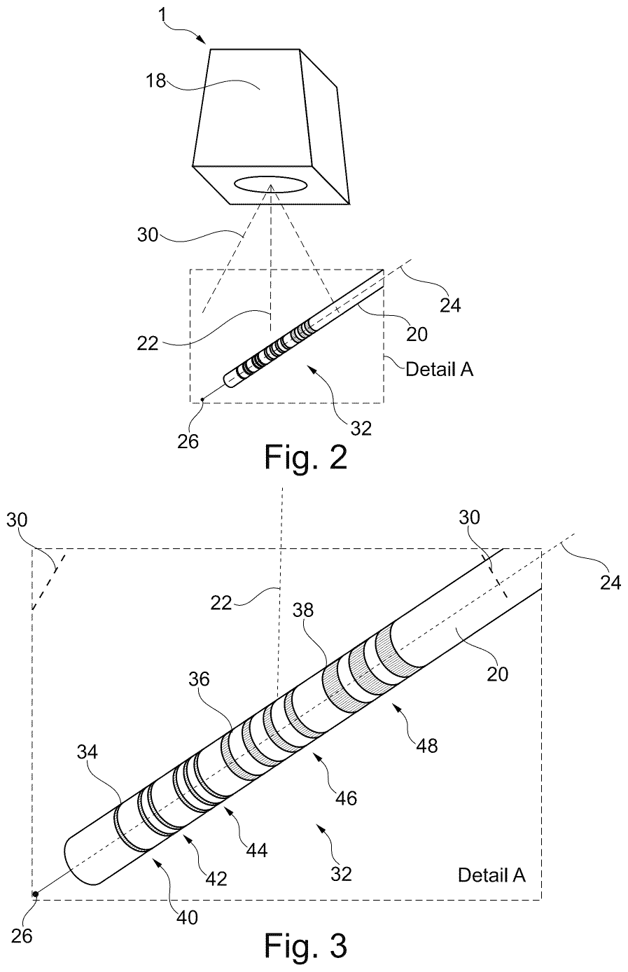

FIG. 2 shows the visualization system 18 with its optical axis 22 and the instrument 20 with its working axis 24 according to FIG. 1 in a perspective view. The visualization system 18 has a field of view 30 which fans out, in which the instrument 20 is at least partially visible and its working point 26 is visibly positioned. During the intervention, the visualization system 18, for example the surgical microscope, continuously creates a real-time, intracorporeal image of the intervention region of the patient P and provides it digitally.

The instrument 20 has a shaft whose surface is formed by a standard geometry and, in the specific configuration example, by a cylinder with a circular shape. At a distal end portion of the shaft adjacent to the working point 26, a surface portion there has a reference marker 32 formed as an optical pattern. The reference marker 32 is adapted so that its optical, imaging detection may be evaluated using an image-processing algorithm for machine vision, so that the position of the working point 26 and the orientation of the working axis 24 may be determined from this.

According to the disclosure, the optical detection is performed by the visualization system 18 using its image, instead of using a separate 3D or 2D navigation camera system, as is common in conventional solutions of the prior art.

FIG. 3 shows a detail A defined according to FIG. 2, which shows an enlarged view of a distal surface portion of the instrument 20, which has the pattern 32, in the area of the working point 26. The pattern 32 is subdivided into individual elements 34, 36 and 38 spaced along the working axis 24, only some of which are representatively provided with the corresponding reference sign in FIG. 3 for the sake of clarity. Each individual one of the elements 34, 36 and 38 has a predetermined distance from the working point 26, which is stored in particular in the control unit 28. According to FIG. 3, the pattern 32 has a variation in the direction of the working axis. In the specific configuration example, the variation consists in the fact that a respective width or extension of the elements 24, 36 and 38, and in some cases also their distance from each other, are different in the stated direction. For example, the elements 38 are the widest and the elements 32 the thinnest, while the width of the elements 36 is in the middle range. All elements 34, 36 and 38 are combined into groups 40, 42, 44, 46, 48, wherein group 40 contains only one element 34, group 42 contains two elements 34, group 44 contains three elements 34, group 46 contains four elements 36, and group 48 contains three elements 38. All elements 34, 36 and 38 extend fully and rotationally symmetrically on the surface portion of the instrument 32. Due to the fact that the optically detectable pattern 32 extends in the aforementioned manner along the surface of the instrument 20, it represents a reference marker formed as a 3D object, which is optically detectable analogous to a conventional rigid body. In contrast, however, its advantage is that it does not require any additional installation space and does not have any additional weight. In addition, it cannot obstruct the line of sight, as it is provided on the surface of the instrument 20, which is already arranged or to be arranged in the field of view.

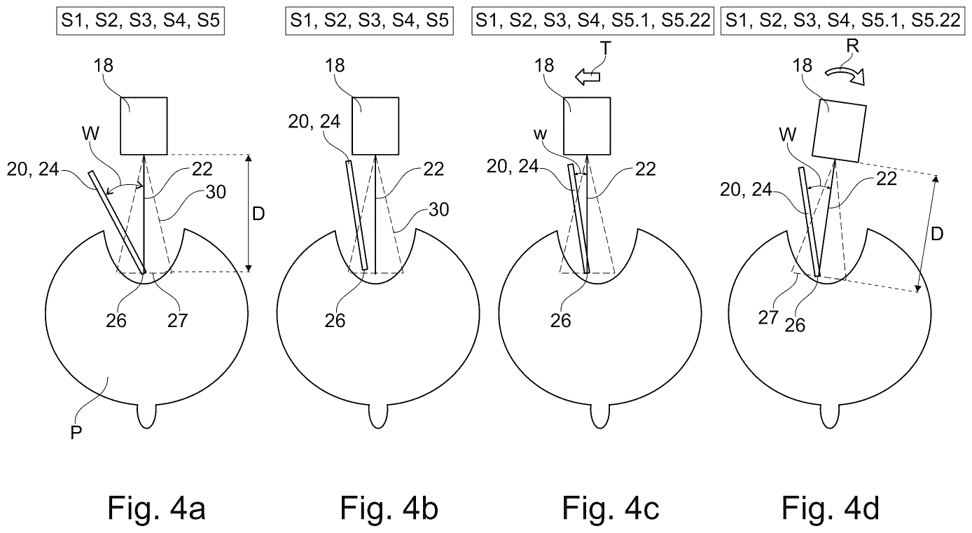

FIGS. 4a to 4d show steps of a method according to the disclosure with the surgical assistance system according to FIGS. 1, 2 and 3.

FIG. 4a shows an intended initial state or basic state in which the visualization system 18 fulfils a predetermined target with respect to its position in space, the orientation of its optical axis 22 and its hyperfocal distance D in relation to the position of the working point 26 and the orientation of the working axis 24 of the instrument. In this state, the following criteria of the target are fulfilled: the working point 26 is arranged in the center of the field of view 30, the working point 26 coincides with the focal distance D and is arranged in an image plane 27. In addition, the optical axis 22 has the predetermined angle W relative to the working axis 24.

In principle, the optical detection of the optical pattern 32 described above takes place continuously via the creation of the image by the visualization system 18. On the basis of the image, the tracking system according to the disclosure determines the position of the working point 26 and the orientation of the working axis 24 by image-processing evaluation of the image and the pattern 32 contained therein using a machine vision algorithm. The control unit 28 continuously checks whether the visualization system 18 fulfils the above-mentioned target, which is the case in the basic state according to FIG. 4a.

FIG. 4b shows a state in which the instrument 20 is deflected from the position and orientation described in FIG. 4a. Accordingly, the tracking system according to the disclosure determines the changed position and orientation of the working point 26 and the working axis 24. As a result, the control unit 28 determines corresponding deviations of the field of view 30, of the orientation of the optical axis 22 and of the hyperfocal distance D from the above-mentioned target.

Depending on the deviations, the control unit 28 determines a necessary translation T and rotation R in one or more translational or rotational degrees of freedom in order to compensate for the deviation from the target by controlling the robot.

According to FIG. 4c, the robot is initially controlled via the control unit 28 in such a way that the visualization system 18 is moved solely translationally, so that the working point 26 is initially moved to the center of the field of view 30 and a coincidence of the working point 26 with the hyperfocal distance D is established.

In the configuration example shown, the robot is controlled sequentially with regard to translation T and rotation R; alternatively, it may of course be synchronized so that the necessary translation T and rotation R are performed simultaneously.

After the sequentially executed translation T according to FIG. 4c, the optical axis 22 and the working axis 24 continue to have a smaller angle w that deviates from the target W. The angular deviation from the target W is subsequently compensated for as shown in FIG. 4d by the control unit 28 controlling the robot in such a way that it performs the necessary rotation R.

The compensation of the deviation from the target via the necessary translation T and rotation R may be carried out automatically and constantly or continuously, as described, for example, with reference to FIGS. 4a to 4d, or it may be carried out only selectively on request. A request signal required for this may, for example, be triggered by a user by pressing an actuating element, such as a pedal or a button, which is arranged in the user's field of action. For example, the instrument 20 may have such an actuating element as an operating interface. Alternatively, the request signal may be triggered by a gesture or a voice command.

The predetermined angle W may be preset depending on the user's preferences and adapted or changed as required. The angle W prevents the shaft of the instrument 20 from blocking or partially blocking the field of view 30 of the microscope to an excessive extent.

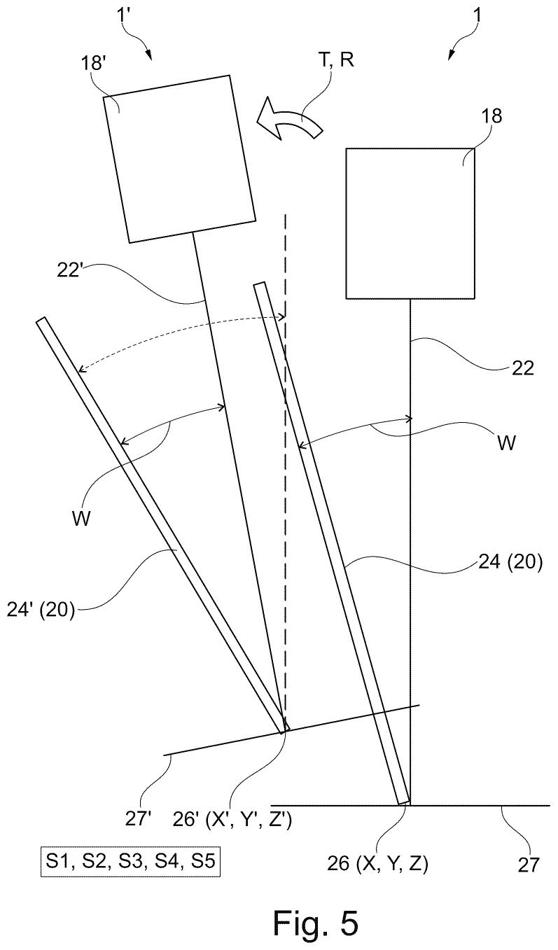

FIG. 5 shows an enlarged view of the process outlined above. The basic state of the assistance system 1 is represented in FIG. 5 to the right of the visualization system 18, the optical axis 22, the image plane 27 arranged orthogonally to the optical axis 22, the working axis 24 of the effector 20, the working point 26 in its coordinates X, Y, Z and. The optical axis 22 is angled towards the working axis 24 with the predetermined angle W according to the target, the working point 26 coincides with the hyperfocal distance and is therefore located in the center of the field of view and in the image plane 27.

A position and orientation of the instrument 20 as shown in FIG. 5 is changed to a new position of the working point 26′ with its new coordinates X′, Y′ and Z′, which corresponds to a translation T, and to a new orientation of the working axis 24′ of the instrument 20′, which corresponds to a rotation R.

The visualization system 18 continuously creates the image in which the transformation of the instrument 20 (translation T and rotation R) is shown in the optically detected change or distortion of the pattern 32.

The tracking system determines the new pose or orientation of the instrument 20 by processing the image, i.e. using the machine vision algorithm, and makes it available to the control unit 28.

From this, the control unit 28 determines the translation T and rotation R of the visualization system 18 required to achieve the target. As soon as the operator triggers the readjustment of the visualization system 18 via a suitable request signal, the control unit 28 controls the robot in such a way that it moves the assistance system 1′ into the constellation represented by the reference signs 1′, 18′, 22′, 24′, 27′.

### LIST OF REFERENCE SIGNS

1 surgical assistance system
2 robot
4 robot base
6,8,10 robot-arm segment
12, 14 hinge
16 end portion of robot arm
18 visualization system
20 effector
22 optical axis
24 working axis
26 working point
27 image plane
28 control unit
30 field of view
32 optical reference marker
S1 Step tracking visualization system
S2 Step creating image
S3 Step optically detecting reference marker
S4 Step determining pose of effector
S5, S5.1, S5.21, S5.22 Step controlling visualization system

1-16. (canceled)

17. A surgical assistance system comprising:
a robot for use for a surgical intervention;
a robot base as a local link point of the robot;
a robot arm that is moveable and connected to the robot base, the robot arm comprising at least one robot-arm segment;
a visualization system connected to the robot arm and adapted to create and provide at least one image in real-time;
a surgical effector comprising a working axis and a working point arranged on or near the working axis, the surgical effector arranged or arrangeable in a field of view of the visualization system;
an optical reference marker arranged on the surgical effector in a predetermined position with respect to the working point, the optical reference marker adapted to determine a position of the working point and/or an orientation of the working axis based on optical detection, wherein the optical reference marker is formed by an optical pattern that extends along at least a surface portion of the surgical effector;
a tracking system adapted to determine the position of the working point and/or the orientation of the working axis as a function of the optical pattern; and
a control unit adapted to control a position of the visualization system and/or an orientation of an optical axis of the visualization system and/or a hyperfocal distance of the visualization system as a function of the position of the working point and/or of the orientation of the working axis,
the control unit being further adapted to control the position of the visualization system and/or the orientation of the optical axis of the visualization system and/or the hyperfocal distance of the visualization system as a function of the position of the working point and/or of the orientation of the working axis such that, when the position of the visualization system is fixed, the hyperfocal distance is set to the working point, or that when the hyperfocal distance is fixed, the position of the visualization system is set such that the hyperfocal distance coincides with the working point.

18. The surgical assistance system according to claim 17, wherein the control unit is adapted to move the visualization system in a tracking mode such that the position of the working point of the surgical effector coincides with a center point or a predefined point of the at least one image, so that the visualization system follows the working point of the surgical effector, and so that the optical axis has a predetermined angle to the working axis and maintains the predetermined angle during movement of the surgical effector.

19. The surgical assistance system according to claim 17, wherein the control unit is adapted to constantly/continuously control the visualization system with respect to a position and/or orientation of the optical axis of the visualization system and/or a hyperfocal distance of the visualization system or to control the visualization system on request/input.

20. The surgical assistance system according to claim 17, wherein the control unit is adapted to determine a flat band spanned by the optical axis and a new working axis, and to define a new optical axis in the flat band with a predetermined angle to the new working axis.

21. The surgical assistance system according to claim 17, wherein the surgical effector is configured with a fixed, non-interchangeable functional unit, on which the working point is arranged.

22. The surgical assistance system according to claim 17, wherein the surgical effector has an interchangeable holder, into which one of several possible functional units is selectively insertable, on which the respective working point is arranged, wherein the interchangeable holder comprises the optical pattern.

23. The surgical assistance system according to claim 22, wherein the optical pattern and/or portions of the optical pattern has or have a respectively predetermined distance from the working point.

24. The surgical assistance system according to claim 17, wherein the optical pattern has a variation at least in a direction of the working axis.

25. The surgical assistance system according to claim 17, wherein the optical pattern has elements that are spaced apart in a direction of the working axis, wherein a distance of the elements from each other and/or a respective extension of the elements in the direction of the working axis has a variation.

26. The surgical assistance system according to claim 25, wherein at least some of the elements are combined to groups within which the elements are the same with respect to their distance from each other and/or their respective extension in the direction of the working axis.

27. The surgical assistance system according to claim 26, wherein the distance of the elements from each other and/or an extension of the elements across the groups has or have a variation.

28. The surgical assistance system according to claim 17, wherein the optical pattern extends completely around the working axis.

29. The surgical assistance system according to claim 17, wherein the optical pattern is at least partially rotationally symmetrical to the working axis.

30. The surgical assistance system according to claim 17, wherein the visualization system is adapted to provide the at least one image to the tracking system, and wherein the tracking system is adapted to determine the position of the working point and/or of the orientation of the working axis of the surgical effector as a function of the at least one image.

31. The surgical assistance system according to claim 17, wherein the surgical effector is a manually guided surgical instrument.

32. A computer-implemented method for controlling a position and/or an orientation of an optical axis and/or of a hyperfocal distance of a visualization system as a function of a surgical effector, in a surgical intervention, the computer-implemented method comprising the steps of:
creating and providing at least one image in real time of an intervention region by a visualization system connected to a robot arm of a surgical robot, wherein a working point of a surgical effector is arranged or arrangeable in a field of view of the visualization system;
optically detecting an optical reference marker, or at least a portion of the optical reference marker, wherein the optical reference marker is formed by an optical pattern that extends along at least a surface portion of the surgical effector, the optical pattern being arranged on the surgical effector in a predetermined position with respect to the working point, such that a detection of the optical pattern is used to determine a position of the working point and/or an orientation of a working axis;
determining the position of the working point and/or of the orientation of the working axis as a function of the optical pattern, by a tracking system; and
controlling the visualization system with respect to a position and/or an orientation of an optical axis of the visualization system and/or a hyperfocal distance of the visualization system as a function of the position of the working point and/or of the orientation of the working axis, via a control unit,
wherein the step of controlling the visualization system comprises the steps of:
driving the surgical robot in such a way that a center or center point of a field of view or of the at least one image or a predefined point of the field of view or of the at least one image is moved to the working point, and that the optical axis is set to a predetermined angle to the working axis; and
a further step comprising:
setting the hyperfocal distance at a predetermined distance of the visualization system to the working point such that the working point is focused or coincides with the hyperfocal distance, or
setting a distance of the visualization system at predetermined, fixed hyperfocal distance to the working point such that the working point is focused or coincides with the hyperfocal distance.

33. The computer-implemented method according to claim 32, wherein the step of controlling the visualization system takes place continuously or only on request as a function of a determined position of the working point and/or of a determined orientation of the working axis.

34. The computer-implemented method according to claim 32, wherein the step of controlling the visualization system is performed as a function of the position of the working point and/or of the orientation of the working axis as a function of a deviation of the position and/or of the orientation of the optical axis and/or of the hyperfocal distance of the visualization system from a target.

35. The computer-implemented method according to claim 34, wherein the target comprises that the working point is visible in a predetermined area of the field of view and/or is depicted in the at least one image, and/or that the working point coincides with the hyperfocal distance, and/or that the optical axis has a predetermined angle to the working axis.

36. A computer-readable storage medium comprising instructions which, when executed by a computer, cause the computer to perform the computer-implemented method according to claim 32.
